(19) United States
(12) Patent Application Publication
(10) Pub. No.: US 2024/0225055 A1
(43) Pub. Date: Jul. 11, 2024

(54) ARONIA BERRY COMPOSITIONS AND METHODS OF MAKING SUCH COMPOSITIONS

(71) Applicant: NUtech Ventures, Lincoln, NE (US)

(72) Inventors: Changmou Xu, Lincoln, NE (US); Rui Huang, Lincoln, NE (US); Xiaoqing Xie, Lincoln, NE (US)

(21) Appl. No.: 18/562,795

(22) PCT Filed: May 20, 2022
(86) PCT No.: PCT/US2022/072484

§ 371 Date: Nov. 20, 2023

Related U.S. Application Data
(60) Provisional application No. 63/201,975, filed on May 20, 2021.

(51) Int. Cl. A23L 2/02; A23L 2/56; A23L 29/25

(52) U.S. Cl. CPC A23L 2/02; A23L 2/56; A23L 29/25

(57) ABSTRACT

The present disclosure provides suitable ingredients, added amounts, and introducing procedures (methods) for mitigating the astringency intensity, increasing the palatability, improving the physical stability, and protecting/maintaining the phenolic compounds from degradation or flocculation during storage (effects) for aronia berry compositional system. In preferred forms, the compositions of this disclosure include gum arabic and the preferred amounts thereof are based on the ratio of gum arabic to total proanthocyanidin content (TPAC) (w/w) in aronia berry compositions.

### CROSS REFERENCE TO RELATED APPLICATIONS

This application claims the priority benefit of U.S. Provisional Patent Application Ser. No. 63/201,975, filed on May 20, 2021, the teachings and contents of which are hereby incorporated by reference.

### STATEMENT REGARDING FEDERALLY SPONSORED RESEARCH & DEVELOPMENT

This invention was made with government support under AM170200XXXXG008/(NDA #18-13-352) awarded by the U.S. Department of Agriculture. The government has certain rights in the invention.

### BACKGROUND OF THE INVENTION

The field of the invention relates generally to aronia berry compositions and methods of making compositions comprising at least a portion of aronia berry. Aronia berry is a superberry, rich in antioxidants (anthocyanins, proanthocyanidins, and total polyphenols), which are significantly richer than many other berries. Therefore, aronia berry is being grown as a specialty crop in the Midwest since around 2010 and throughout the U.S. with over 1,500 acres. However, the horribly astringent taste of aronia berry and its juice (because of the very high polyphenols) decreases consumer preference of aronia berry products, which has become its number one problem and ultimately prevented the development of the aronia berry industry. Aronia berry juice also suffers from a lack of stability. For example, when it is packaged in a beverage bottle, polyphenols in the juice will aggregate with each other and cause precipitation, which ultimately results in the loss of nutrients (this problem can be found in commercial aronia juice products in a supermarket). Accordingly, what is needed in the art are compositions and methods which decrease the astringency of aronia berry products. What is further needed are ingredients, products, and/or ways to improve the taste of aronia berry products. What is still further needed are compositions and methods for increasing the stability and preference of aronia berry products.

### BRIEF DESCRIPTION OF THE INVENTION

The present application solves the problems inherent in the prior art and provides compositions and methods for increasing the palatability, taste preference, color preference, and/or taste profile of compositions containing aronia berry or aronia berry juice; decreasing the astringency of aronia berry or aronia berry juice as well as products containing such components; and increasing the stability of compositions containing or comprising aronia berry juice. Additionally, the present application increases the stability, both in terms of reducing or inhibiting precipitation of the ingredients in compositions containing aronia berry as well as in maintaining the nutrient profile of such compositions.

One aspect of this disclosure provides methods and ingredients for increasing the palatability or taste (consumer) preference of compositions containing aronia berry or aronia berry juice. In some forms, the palatability is increased by decreasing or mitigating the astringency. In some forms, the sourness is mitigated. In some forms, the bitterness is mitigated. In some forms, at least two of astringency, sourness, and bitterness are mitigated. In some forms, astringency, sourness, and bitterness are all mitigated. Preferably, the mitigation of astringency, sourness, and/or bitterness is in comparison to a composition that does not contain the ingredients or is not processed by or made using the methods disclosed herein. In some forms, the mitigation reduces astringency, and/or sourness, and/or bitterness by at least 10, 20, 30, 40, 50, 60, 70, 80, 90, 95% or more. In some forms, the percentage reduction is determined using a scale of astringency and/or sourness and/or bitterness and is based on a comparison with a composition that does not contain the ingredients or is not processed by or made using the methods disclosed herein. In some forms, the percentage reduction is determined based on a comparison with a composition that does not contain the ingredients or is not processed by or made using the methods disclosed herein using the number percentage of people subjectively rating the astringency and/or sourness and/or bitterness as lower. In some preferred forms, the composition includes an ingredient in addition to aronia berry and/or aronia berry juice. In some forms, the ingredient includes gum arabic. In some preferred methods, the methods include the step of adding gum arabic to the aronia berry composition or adding the aronia berry composition to the gum arabic. In some preferred forms, the aronia juice composition is added to gum arabic. In some forms, the aronia juice was added gradually to gum arabic. In some forms, the aronia juice was added to gum arabic gradually with stirring. In some forms, the gum arabic is in powder form. In some forms, the gum arabic comprises 1.5-4% w/w of the final composition including 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0% and all amounts therebetween. It is understood that these amounts can refer to the amount of gum arabic in the final composition or the amount of gum arabic relative to the amount of aronia berry or aronia berry juice. Preferably the amount of gum arabic is between 1.8-2.4% w/w. In some forms, the amount of aronia berry in the composition, excluding any gum arabic, is between 10-100% including 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9, and 100% w/w. In some forms the amount of aronia berry in the composition is between 82-88%, more preferably between 84-86%, and still more preferably about 85%. In some forms, the aronia berry composition is aronia berry juice. In some forms, the aronia berry juice is present in the composition in an amount greater than 75% including 75, 76, 77, 78, 79, 80, 81, 82, 83, 84, 85, 86, 87, 88, 89, 90, 91, 92, 93, 94, 95, 96, 97, 98, 99, or more. In some forms, the present disclosure provides a method of reducing the astringency of an aronia berry composition comprising the step of combining the composition with an amount of gum arabic. In some forms, the aronia berry composition and the gum arabic are combined using a method selected from the group consisting of adding the aronia berry composition slowly to gum arabic by stirring, adding gum arabic to the aronia berry composition with very fast and vigorous mixing, adding the aronia berry composition slowly and gradually to the gum arabic with continuous stirring, or dissolving gum arabic in the water first and then adding aronia berry composition gradually with stirring. In some forms, the gum arabic is in a form selected from the group consisting of powder or aqueous liquid. In some forms, the aronia berry composition is aronia berry juice. In some forms, the aronia berry juice is present in the composition in an amount of greater than 75% w/w. In some forms, the aronia berry juice comprises 84-86% w/w of the composition. In some forms, the aronia berry composition comprises up to 4% w/w gum arabic. In some forms, the ratio of gum arabic to total proanthocyanidins content (TPAC) (w/w) in said aronia berry juice is at least 22.15. In some forms, the TPAC is at least 22.15-23.13. In some forms, the TPAC is 22.15, 22.25, 22.35, 22.45, 22.55, 22.65, 22.75, 22.85, 22.95, 23.05, 23.13, 23.23, 23.33, 23.43, 23.53, 23.63, 23.73, 23.83, 23.93 or more.

Another aspect of this disclosure provides methods and ingredients for protecting and/or maintaining the stability and/or nutrients of aronia berry juice products. In some forms, the compositions of the disclosure remain stable for 10, 20, 30, 40, 50, 60, 70, 80, 90, 95%, or longer in comparison to compositions not made in accordance with this disclosure. In some forms, the comparison measures the amount of nutrient loss or retention of at least 1, 2, 3, 4, 5, 6, 7, 8, 9, 10, or more nutrient(s) in the compositions. In some forms, the total phenolic content is compared to determine the stability. In some forms, the comparison measures the amount of precipitation in the compositions. In some forms, the amount of precipitation is measured using light transmission through different parts of the composition. For example, a comparison of the light transmission through the top ¼ of the composition compared with the light transmission through the bottom ¼ of the composition. As can be appreciated, compositions experiencing greater precipitation will have a greater disparity in light transmission between the top and bottom portions of the composition. In some forms, the turbidity of compositions in accordance with this disclosure is compared with compositions that are not made in accordance with this disclosure or that do not have the ingredient, preferably gum arabic. In some forms, aggregation and/or flocculation is compared. In some preferred forms, the ingredient includes gum arabic. In some preferred methods, the methods include the step of adding gum arabic to the composition. In some preferred forms, the aronia juice composition is added to gum arabic. In some forms, the aronia juice was added gradually to gum arabic. In some forms, the aronia juice was added to gum arabic gradually with stirring. In some forms, the gum arabic is added to the aronia berry juice composition and vortexed. In some forms, the gum arabic comprises up to 4% w/w of the aronia berry composition. In some forms, the gum arabic comprises between 0.2%-4.0% w/w including 0.2. 0.3, 0.4, 0.5, 0.6, 0.7, 0.8, 0.9. 1.0, 1, 1.1, 1.2, 1.3, 1.4, 1.5, 1.6, 1.7, 1.8, 1.9, 2.0, 2. 1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0% and all amounts therebetween. It is understood that these amounts can refer to the amount of gum arabic in the final composition or the amount of gum arabic relative to the amount of aronia berry or aronia berry juice. Preferably the amount of gum arabic is between 1.8-2.4% w/w. In some forms, the amount of aronia berry in the composition is between 10-90% including 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9, and 100% w/w. In some forms the amount of aronia berry in the composition is between 82-88%, more preferably between 84-86%, and still more preferably about 85%. In some forms, the aronia berry composition is aronia berry juice. In some forms, the present disclosure provides a method of increasing the stability of an aronia berry composition comprising the step of combining the composition with an amount of gum arabic. In some forms, the aronia berry composition is added to gum arabic using a method selected from the group consisting of adding the aronia berry composition slowly to gum arabic by stirring, adding gum arabic to the aronia berry composition with very fast and vigorous mixing, adding the aronia berry composition slowly and gradually to the gum arabic with continuous stirring, or dissolving gum arabic in the water first and then adding aronia berry composition gradually with stirring. In some forms, the gum arabic is in a form selected from the group consisting of powder and aqueous liquid. In some forms, the aronia berry composition is aronia berry juice. In some forms, the aronia berry juice is present in the composition in an amount of greater than 75% w/w. In some forms, the aronia berry juice comprises 84-86% w/w of the composition. In some forms, the composition comprises up to 4% w/w gum arabic. In some forms, the ratio of gum arabic to total proanthocyanidins content (TPAC) (w/w) in said aronia berry juice is at least 22.15. In some forms, the TPAC is 22.15, 22.25, 22.35, 22.45, 22.55, 22.65, 22.75, 22.85, 22.95, 23.05, 23.13, 23.23, 23.33, 23.43, 23.53, 23.63, 23.73, 23.83, 23.93 or more. In some forms, the increase in stability is determined by a comparison of the TPAC between an aronia berry composition containing gum arabic and an aronia berry composition that does not contain gum arabic. In some forms, the increase in stability is determined by a comparison of the amount of aggregation or flocculation between an aronia berry composition that contains gum arabic and an aronia berry composition that does not contain gum arabic. In some forms, the increase in stability is determined by a comparison of the content of at least one nutrient between an aronia berry composition that contains gum arabic and an aronia berry composition that does not contain gum arabic.

The present application compares a large number of food ingredients, including various proteins and polysaccharides and demonstrates the unexpected superiority of gum arabic in improving the sensory properties, stability and bioactivity of aronia berry juice. Further, the present application describes the best ratio and encapsulating procedure to apply the gum arabic, which are keys to successfully overcoming the problems inherent in the art. At least some of the problems identified in the prior art are related to the unique polyphenols composition in aronia juice.

Gum arabic is a polysaccharide and common food additive (naturally sourced, Generally Recognized As Safe (GRAS)) used as thickener, emulsifier, stabilizer, flavoring agent, or dietary fiber in many food products. However, no study has been reported on its effectiveness in mitigating the taste (astringency, sourness and/or bitterness) and protecting the stability and nutrients of aronia berry juice products. Although gum arabic has similar effects in other food products (as a stabilizer or flavoring agent), such effects are unpredictable. Additionally, the amount of gum arabic needed as well as how it must be applied to, added to, or included in the composition in order to be effective at overcoming the problems inherent in the prior art were also unpredictable and surprising.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIG. 1A is a photograph illustrating the reaction of aronia juice with different concentrations of pectin after centrifugation;

FIG. 1B is a photograph illustrating the reaction of aronia juice with different concentrations of K-carrageenan after centrifugation;

FIG. 1C is a photograph illustrating the reaction of aronia juice with different concentrations of xanthan gum after centrifugation;

FIG. 1D is a photograph illustrating the reaction of aronia juice with different concentrations of CMC-Na after centrifugation;

FIG. 1E is a photograph illustrating the reaction of aronia juice with different concentrations of gum arabic (Seyal) after centrifugation;

FIG. 1F is a photograph illustrating the reaction of aronia juice with different concentrations of gum arabic (Senegal) after centrifugation;

FIG. 2A is a photograph illustrating the physical status/appearance of aronia juice with different concentration of gum arabic;

FIG. 2B is a graph illustrating the turbidity of aronia juice with different concentrations of gum arabic;

FIG. 2C is a graph illustrating the total phenolic content (TPC) of aronia juice with different concentrations of gum arabic;

FIG. 2D is a graph illustrating the total anthocyanins content (TAC) of aronia juice with different concentrations of gum arabic;

FIG. 3A is a graph illustrating the astringency intensity (1: Extremely weak, 9: Extremely strong) and liking (1: Dislike Extremely, 9: Like Extremely) of “75% aronia juice+25% water” vs “aronia juice”;

FIG. 3B is a graph illustrating the astringency intensity (1: Extremely weak, 9: Extremely strong) and liking (1: Dislike Extremely, 9: Like Extremely) of “2.3% gum arabic+97.7% juice” (before transformation recipe) vs “aronia juice;

FIG. 3C is a graph illustrating the astringency intensity (1: Extremely weak, 9: Extremely strong) and liking (1: Dislike Extremely, 9: Like Extremely) of “2.3% gum arabic+97.7% juice” (before transformation recipe) vs “2% gum arabic+13% water+85% juice” (after transformation recipe);

FIG. 3D is a graph illustrating the astringency intensity (1: Extremely weak, 9: Extremely strong) of aronia juice with gum arabic from 2% to 2.6% (after transformation recipe) (N=4);

FIG. 3E is a graph illustrating the liking (1: Dislike Extremely, 9: Like Extremely) of aronia juice with gum arabic from 2% to 2.6% (after transformation recipe) (N=4);

FIG. 4A is a photograph illustrating the storage stability by the physical status/appearance of aronia juice with different concentrations of gum arabic (from 2% to 2.6%, after transformation recipe) at the first day;

FIG. 4B is a set of photographs illustrating the storage stability evidenced by the physical status/appearance of aronia juice with different concentrations of gum arabic (from 2% to 2.6%, after transformation recipe) at a week (7 days) later;

FIG. 5A is a graph illustrating the validation of the effects of 2.2%, 2.3%, and 2.4% concentrations of gum arabic (after transformation recipe) on the astringency intensity (1: Extremely weak, 9: Extremely strong) of aronia juice;

FIG. 5B is a graph illustrating the validation of the effects of 2.2%, 2.3%, and 2.4% concentrations of gum arabic (after transformation recipe) on the liking (1: Dislike Extremely, 9: Like Extremely) of aronia juice;

FIG. 6A is a photograph illustrating the physical status/appearance of the composition of aronia juice and 2.3% of gum arabic (after transformation recipe) after “adding gum arabic to aronia juice” vs “adding aronia juice slowly to gum arabic with stirring,” where the left tube represents “adding gum arabic to aronia juice” (2.3% gum arabic, after transformation recipe), i.e., 85 g of aronia juice mixing with 13 g of water first, followed by adding 2 g of gum arabic (100 g in total); and the right tube represents “adding aronia juice slowly to gum arabic with stirring” (2.3% gum arabic, after transformation recipe), i.e., adding 85 g of aronia juice slowly and gradually to 2 g of gum arabic with stirring first, followed by adding 13 g of water (100 g in total);

FIG. 6B is a photograph illustrating the physical status/appearance of the composition of aronia juice and 2.3% of gum arabic (after transformation recipe) after “dissolving gum arabic in the water first and then adding aronia juice” vs “adding aronia juice slowly to gum arabic with stirring, where the left tube represents “dissolving gum arabic in the water first and then adding aronia juice” (2.3% gum arabic, after transformation recipe), i.e., 2 g of gum arabic was dissolved in 13 g of water first, followed by adding 85 g of aronia juice slowly and gradually with stirring (100 g in total); and the right tube represents “adding aronia juice slowly to gum arabic with stirring” (2.3% gum arabic, after transformation recipe), i.e., adding 85 g of aronia juice slowly and gradually to 2 g of gum arabic with stirring first, followed by adding 13 g of water (100 g in total);

FIG. 7A is a photograph illustrating the physical status/appearance of the composition of aronia juice with 2.2%, 2.3%, and 2.4% of gum arabic (after transformation recipe) after pasteurization and being stored for a week (7 days) at room temperature (25° C.);

FIG. 7B is a set of photographs illustrating the physical status/appearance of the composition of aronia juice with 2.2%, 2.3%, and 2.4% of gum arabic (after transformation recipe) after pasteurization and being stored for a week (7 days) in the fridge (4° C.);

FIG. 8 is a set of photographs illustrating the physical status/appearance of the composition of aronia juice with 2.3% of gum arabic (after transformation recipe) after pasteurization and being stored for 11 weeks (77 days) at room temperature (25° C.) (left bottle) and in the fridge (4° C.) (right bottle);

FIG. 9 is a graph illustrating the total phenolic content (TPC) of aronia juice during storage after being pasteurized (TT: thermal treated);

FIG. 10A is a graph illustrating the astringency intensity (1: Extremely weak, 9: Extremely strong) of aronia juice with 2.3% gum arabic (after transformation recipe) composition by substituting water with apple juice and grape juice (N=5);

FIG. 10B is a graph illustrating the liking (1: Dislike Extremely, 9: Like Extremely) of aronia juice with 2.3% gum arabic (after transformation recipe) composition by substituting water with apple juice and grape juice (N=5);

FIG. 11 is a graph illustrating the astringency intensity (1: Extremely weak, 9: Extremely strong) of aronia puree before and after introducing 2.5% gum arabic. (N=4, three repeats);

FIG. 12A is a graph illustrating the color liking (1: Dislike Extremely, 9: Like Extremely) of original aronia juice (OAJ) and aronia juice with 2% gum arabic (AJG) (N=60);

FIG. 12B is a graph illustrating the aroma liking (1: Dislike Extremely, 9: Like Extremely) of original aronia juice (OAJ) and aronia juice with 2% gum arabic (AJG) (N=60);

FIG. 12C is a graph illustrating the sweetness liking (1: Dislike Extremely, 9: Like Extremely) of original aronia juice (OAJ) and aronia juice with 2% gum arabic (AJG) (N=60);

FIG. 12D is a graph illustrating the sourness liking (1: Dislike Extremely, 9: Like Extremely) of original aronia juice (OAJ) and aronia juice with 2% gum arabic (AJG) (N=60);

FIG. 12E is a graph illustrating the bitterness liking (1: Dislike Extremely, 9: Like Extremely) of original aronia juice (OAJ) and aronia juice with 2% gum arabic (AJG) (N=60);

FIG. 12F is a graph illustrating the astringency intensity (1: Dislike Extremely, 9: Like Extremely) of original aronia juice (OAJ) and aronia juice with 2% gum arabic (AJG) (N=60);

FIG. 12G is a graph illustrating the astringency liking (1: Dislike Extremely, 9: Like Extremely) of original aronia juice (OAJ) and aronia juice with 2% gum arabic (AJG) (N=60);

FIG. 12H is a graph illustrating the overall liking (1: Dislike Extremely, 9: Like Extremely) of original aronia juice (OAJ) and aronia juice with 2% gum arabic (AJG) (N=60);

FIG. 13A is a photograph illustrating the physical status/appearance of original aronia juice (OAJ, left) and aronia juice with 2% gum arabic (AJG, right) at 0 month;

FIG. 13B is a photograph illustrating the physical status/appearance of original aronia juice (OAJ, left) and aronia juice with 2% gum arabic (AJG, right) at the 6th month (that is after being stored for 6 months).

### DETAILED DESCRIPTION OF THE INVENTION

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Example 1

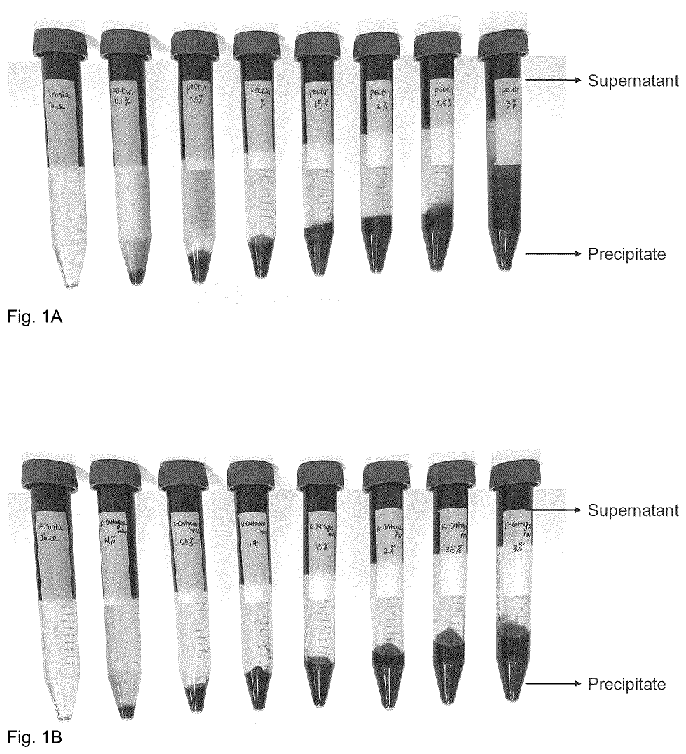

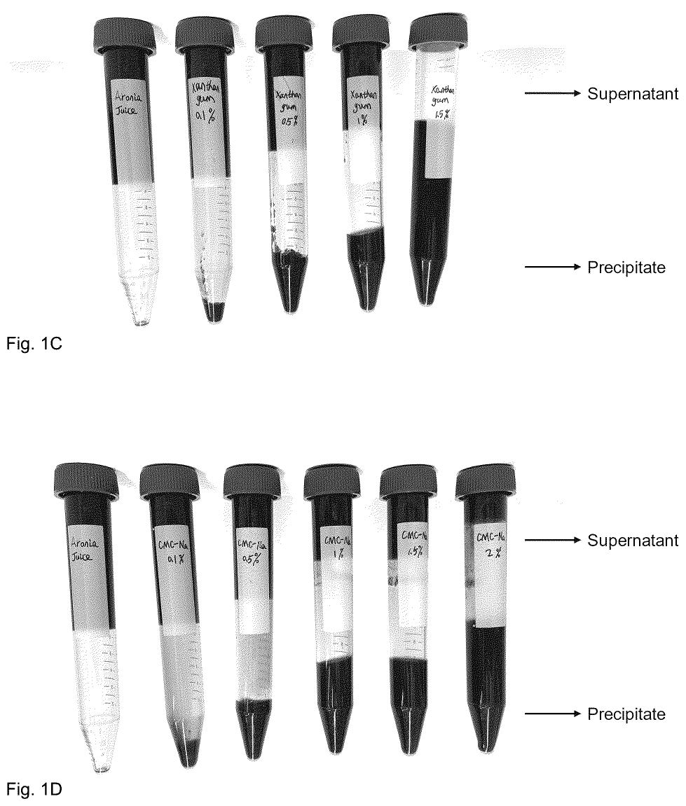

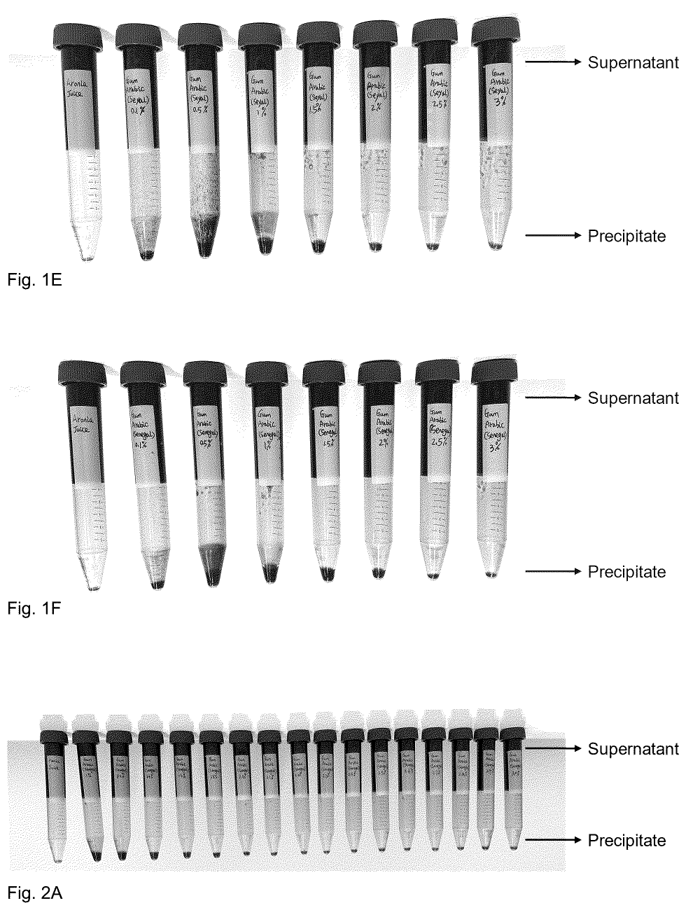

Five polysaccharides, including pectin, κ-carrageenan, xanthan gum, CMC-Na, and gum arabic, were tested for aggregation and flocculation (reflected by photographs). As shown in FIG. 1, pectin, K-carrageenan, xanthan gum, and CMC-Na were not suitable for aronia juice, because after they were mixed with aronia juice, lots of floccules were formed. For gum arabic including Seyal and Senegal, both are suitable for aronia juice systems and compositions as the aggregation and flocculation were decreased in comparison to the other added ingredients, and the concentration should be no less than 1%. Therefore, among five tested polysaccharides (xanthan gum, pectin, κ-carrageenan, CMC-Na, and gum arabic), only gum arabic would not induce intense aggregation or flocculation when added to an aronia berry or aronia juice system.

### Example 2

In this example, the suitable concentration range of gum arabic for aronia juice system was determined.

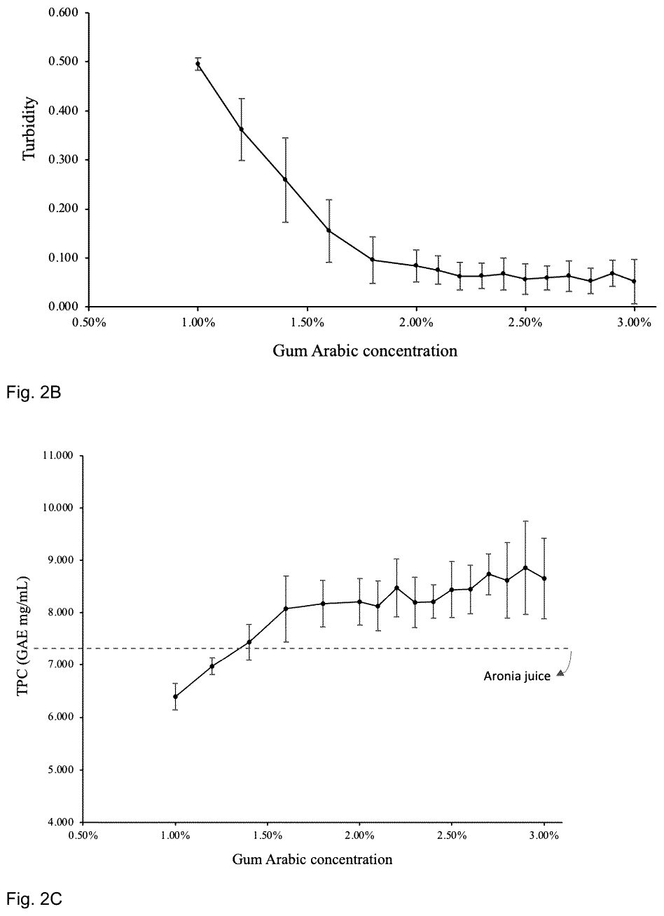

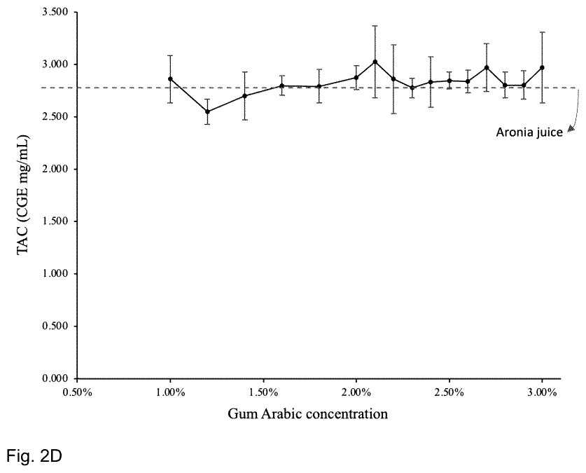

As displayed in FIG. 2(A) and (B), aronia juice with gum arabic in the concentrations from 2% to 3% showed good and similar physical status/appearance and turbidity. As the TPC of aronia juice with no less than 1.6% of gum arabic was even higher than the original aronia juice (FIG. 2(C)), while the gum arabic alone did not show any phenolic content, thus the interaction of gum arabic and phenolic compounds in aronia juice might interfere the Folin-Ciocalteu method. Combining with the results from FIG. 2(D), where the TAC of aronia juice with gum arabic from 2% to 3% was similar to the original juice, and few floccules were separated out from the aqueous system (FIG. 2(A) and (B)), it is suggested that the actual TPC of aronia juice with gum arabic from 2% to 3% was similar to the original juice.

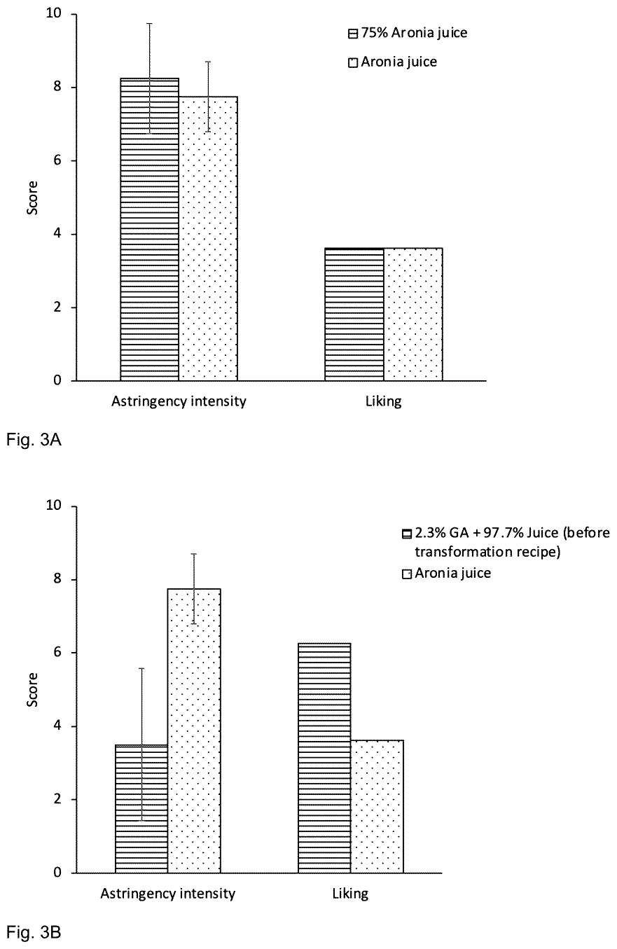

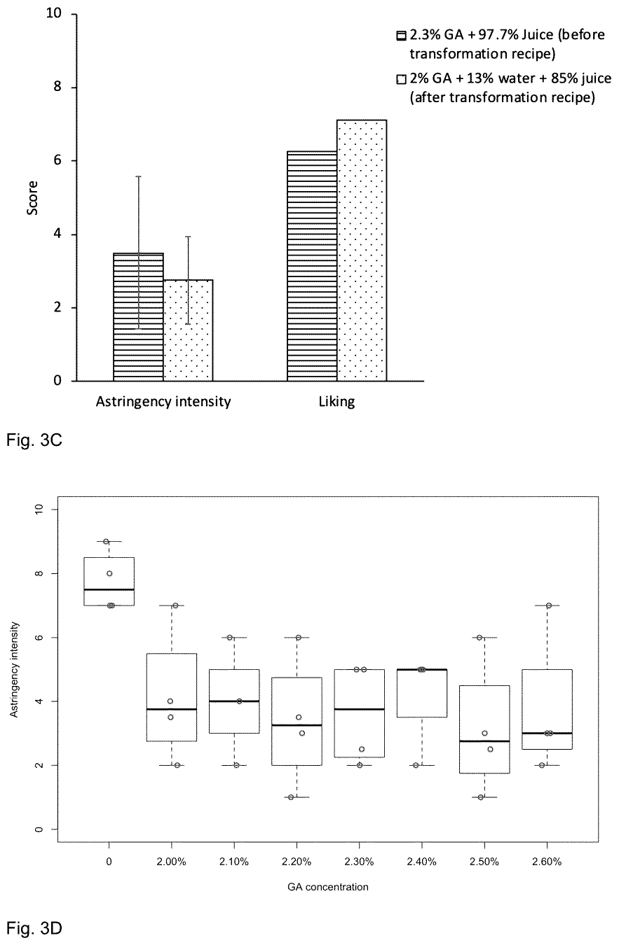

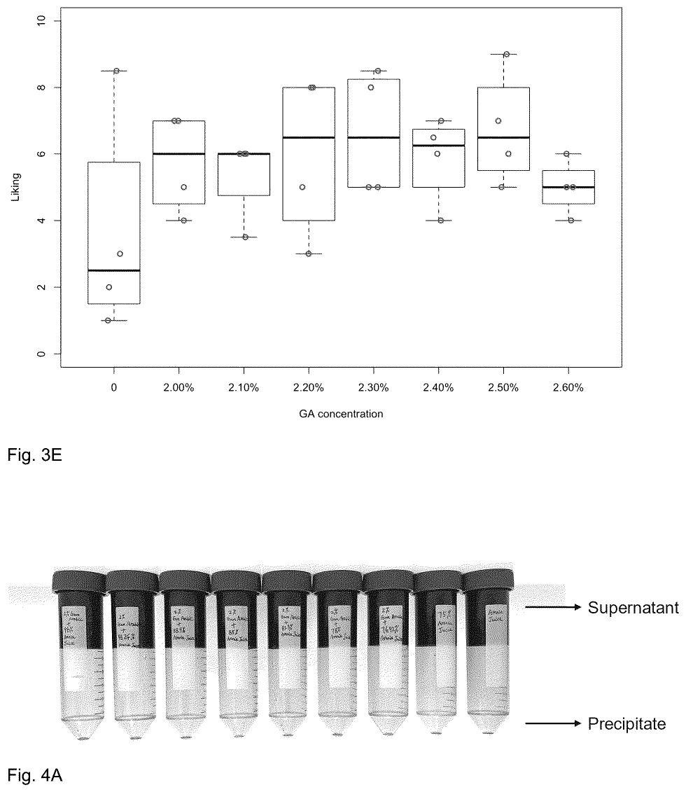

To test the effects of gum arabic on the astringency intensity of aronia juice, we firstly conducted a sensory panel to compare “75% aronia juice+25% water” with “aronia juice”; “2.3% gum arabic+97.7% juice” (before transformation recipe) with “aronia juice”; and “2.3% gum arabic+97.7% juice” (before transformation recipe) with “2% GA+13% water+85% juice” (after transformation recipe). As displayed in FIG. 3(A), dilution of aronia juice from 100% to 75% by water could not effectively change its astringency intensity and palatability. FIG. 3(B) showed that introducing 2.3% gum arabic into aronia juice system could effectively reduce astringency intensity and improve palatability of aronia juice. Based on FIG. 3(C), transforming 2.3% of gum arabic to 2% in compliance with the FDA's policy and at the same time fixing the ratio of gum arabic to aronia juice, the astringency intensity and liking degree of sample did not have notable change, suggesting the transformation method is feasible. Then we evaluated the astringency intensity and palatability of aronia juice with gum arabic from 2% to 2.6% following “after transformation recipe”. As FIG. 3(D) and (E) exhibited, all concentrations of gum arabic from the range of 2% to 2.6% (after transformation recipe) could effectively mitigate the astringency intensity (from over 5 to less than 5 point) and improve the palatability (from less than 5 to over 5 point) of aronia juice.

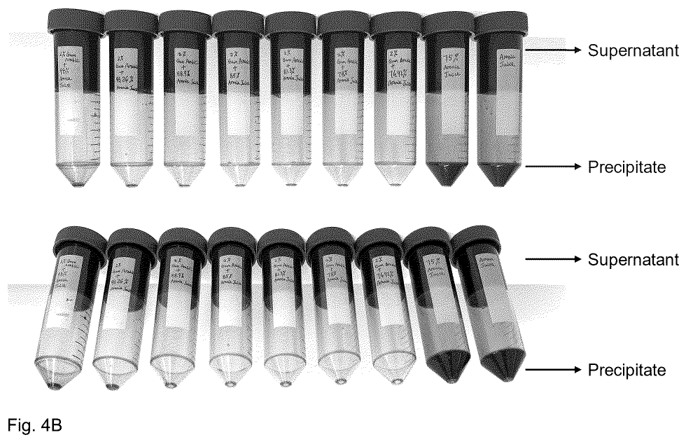

FIG. 4 indicated that when being stored in the fridge for seven days, 100% and 75% aronia juice formed a lot of precipitates; aronia juice with 2% and 2.1% of gum arabic (after transformation recipe) were not very stable and also formed certain floccules; aronia juice with 2.2% of gum arabic (after transformation recipe) formed a little bit floccule but not too bad; aronia juice with 2.3% to 2.6% of gum arabic (after transformation recipe) were stable. Therefore, we speculated that the minimal concentration of introduced gum arabic should be over 2.2% to make the mixture system stable (critical value). We then selected 2.2%, 2.3%, and 2.4% concentrations of gum arabic to validate their effects on the astringency intensity and palatability of aronia juice.

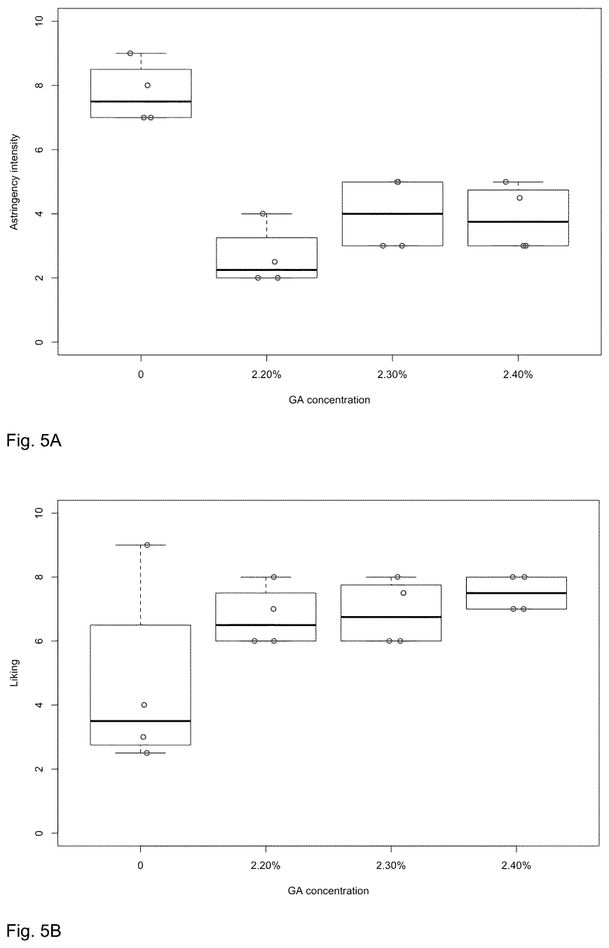

As shown in FIG. 5, we validated that gum arabic at the concentrations of 2.2%, 2.3%, and 2.4% (after transformation recipe) could effectively mitigate the astringency intensity and improve palatability of aronia juice.

### Example 3

In this example, the suitable introducing procedure of gum arabic into aronia juice was described.

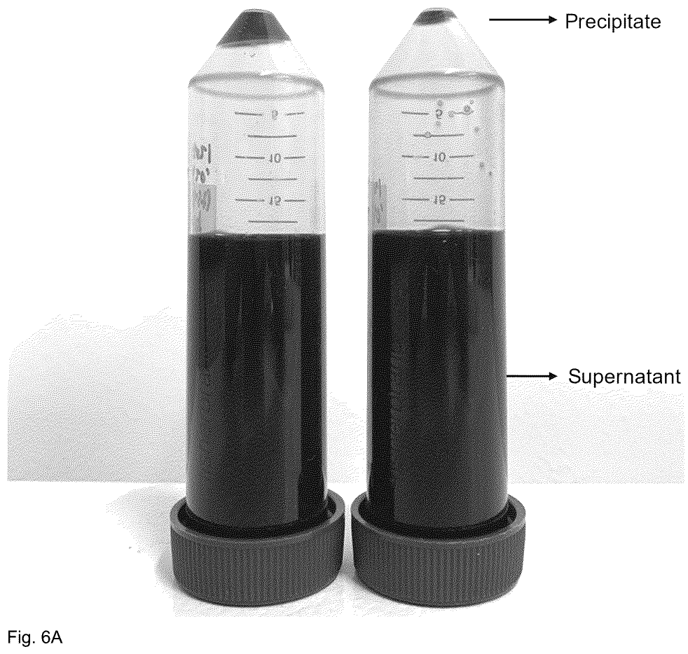

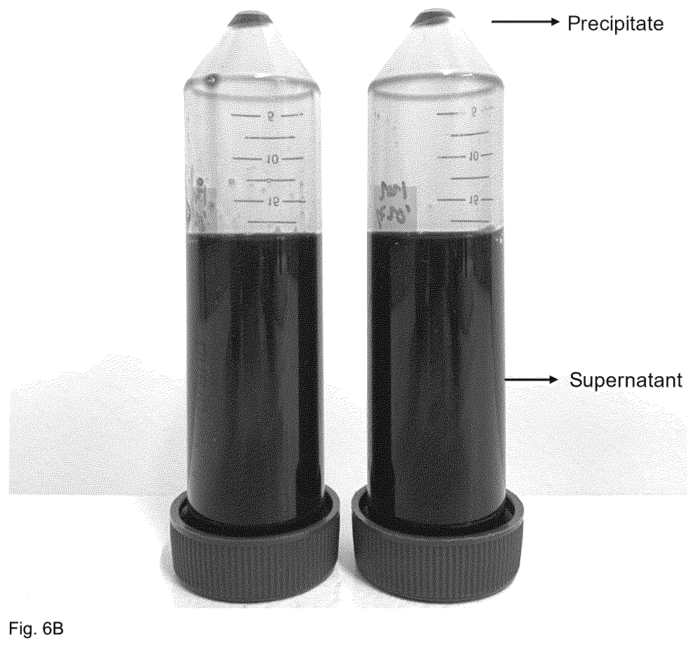

As displayed in FIG. 6(A), when adding gum arabic into aronia juice system, lots of floccules will be formed (left tube), however, when adding aronia juice to gum arabic gradually and slowly with stirring, only few precipitates appeared (right tube). This is in agreement with the results from FIG. 2(A) that when the ratio of gum arabic concentration to aronia juice concentration in the mixture system was lower than a critical value, the floccules would be formed. FIG. 6(B) indicated that gum arabic could be firstly dissolved in water and then mixing with aronia juice (left tube), which had similar final physical status/appearance with the one that adding aronia juice slowly and gradually to gum arabic with stirring (right tube). Both produced only few precipitates. It is useful for practical industrial production. Therefore, we suggested the gum arabic introducing procedure including “adding gum arabic to the aronia juice with very fast and vigorous mixing”, or “adding aronia juice slowly and gradually to the gum arabic with continuous stirring”, or “dissolving gum arabic in the water first and then adding aronia juice gradually with stirring” as preferred.

### Example 4

In this example, the physical stability and total phenolic content (TPC) of aronia juice with or without gum arabic after pasteurization during storage were described, and the preferred addition amount of gum arabic was determined and described.

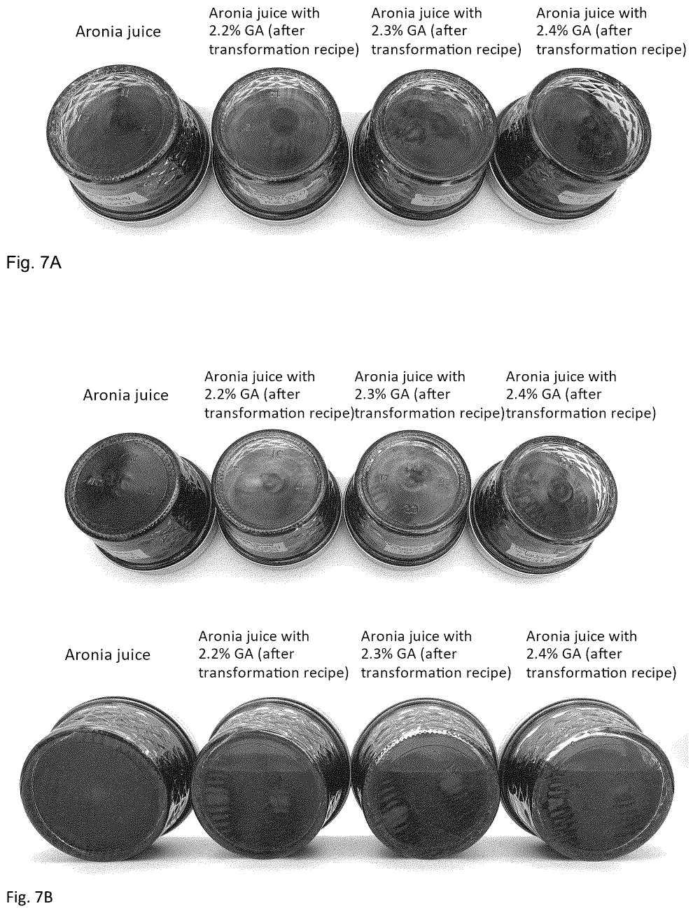

After being stored at room temperature (25° C.( for a week (7 days), aronia juice produced some particles settling at the bottom of the bottle, while for other three samples with the addition of gum arabic from 2.2% to 2.4% concentration (after transformation recipe), the mixture systems were stable and clear (FIG. 7(A)). After being stored in the fridge (4° C.) for a week (7 days), aronia juice forms a lot of precipitates settling at the bottom of the bottle. With the addition of gum arabic, the precipitates could be effectively suppressed. However, aronia juice with 2.2% of gum arabic (after transformation recipe) still produced many floccules, which could be seen on the edge of the bottom. For aronia juice with 2.3% and 2.4% of gum arabic (after transformation recipe), the mixture system was stable and clear (FIG. 7(B)). Therefore, addition of 2.3% and 2.4% of gum arabic (after transformation recipe) would be much better for the storage stability of aronia juice, especially at 4° C.

In the mixture system of aronia juice with 2.3% or 2.4% of gum arabic, there are 97.7% aronia juice and 2.3% gum arabic in the 2.3% gum arabic system, or 97.6% aronia juice and 2.4% gum arabic in the 2.4% gum arabic system (before transformation). The astringency related phenolic compounds in aronia juice majorly belong to proanthocyanidins (based on our previous research). Based on Table 1, total proanthocyanidin content (TPAC) in aronia juice (1) is 1.127+0.110 mg catechin equivalent/mL, i.e. 1.063 mg CE/g juice (the density of aronia juice is 1.06 g/mL). So, the ratios of gum arabic to TPAC are 22.15 (w/w) in the 2.3% gum arabic system and 23.13 (w/w) in the 2.4% gum arabic system, which were the minimum critical amount of gum arabic needed to be introduced, Therefore, preferred added amount of gum arabic is based on the ratio of gum arabic to total proanthocyanidin content (TPAC) (w/w) in aronia juice, which should be 22.15-23.13 or higher, i.e., if we determined the TPAC in aronia juice as 1 g CE by DMAC method, we should at least use 22.15-23.13 g of gum arabic.

TABLE 1
Phenolic profile of three different aronia berry juices

| | Aronia | Aronia | Aronia |
|---|---|---|---|
| | juice (1) | juice (2) | juice (3) |
| TPC (mg GAE/mL) | 7.319 ± 0.353 | 2.953 ± 0.143 | 7.326 ± 0.314 |
| TPAC (mg CE/mL) | 1.127 ± 0.110 | 0.269 ± 0.012 | 1.462 ± 0.138 |
| TAC (mg | 2.781 ± 0.167 | 0.974 ± 0.051 | 1.804 ± 0.044 |
| Cy3Glc/mL) | | | |

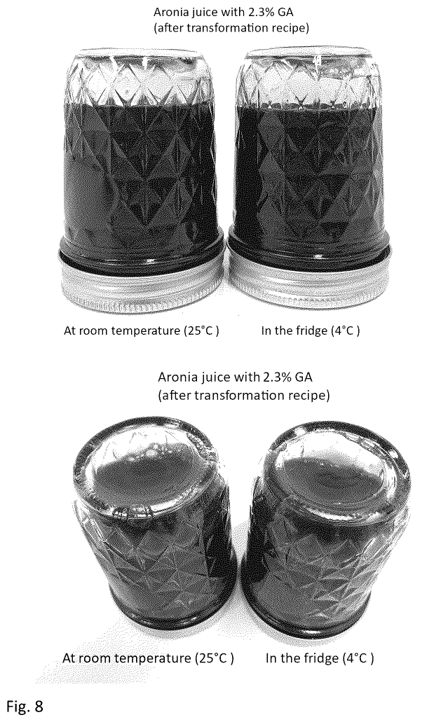

After being stored at room temperature (25° C.) and in the fridge (4° C.) for 11 weeks (77 days), the mixture system of aronia juice with 2.3% of gum arabic (after transformation recipe) was still stable (FIG. 8).

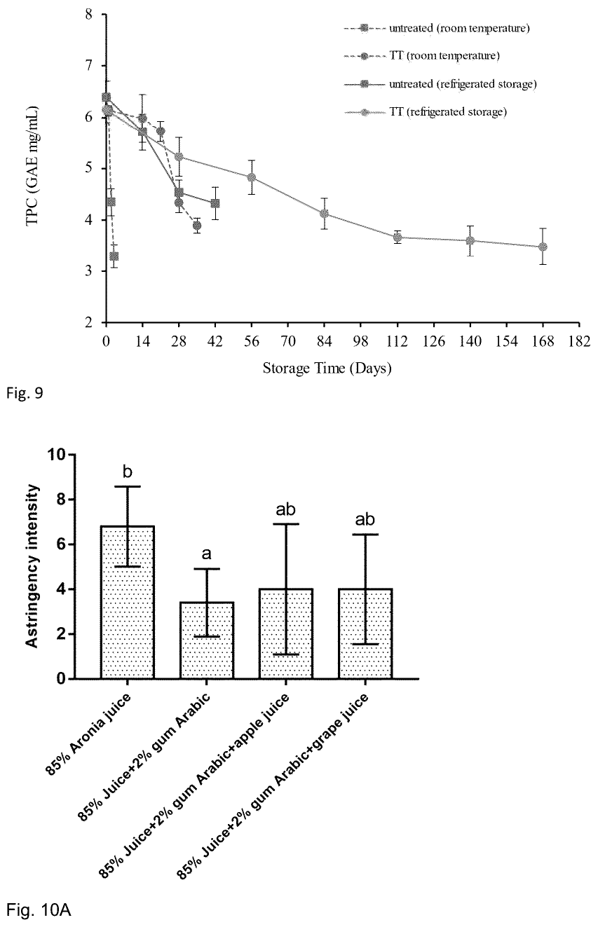

As shown in FIG. 9, the TPC in aronia juice after pasteurization was remarkably declined with the extension of storage time. In contrast, the TPC in aronia juice with 2.3% gum arabic (after transformation recipe) after pasteurization was not decreased notably even after being stored for 11 weeks no matter at room temperature or in the fridge (Table 2). This indicated that the introduction of gum arabic into aronia juice system is beneficial to protect/maintain the phenolic compounds in aronia juice.

TABLE 2
Total phenolic content (TPC) of aronia juice with 2.3% gum
arabic (after transformation recipe) after pasteurization
and being stored for 11 weeks (77 days) at room temperature
(25° C.) (left bottle) and in the fridge (4° C.).

| | After heating | After storage at | After storage at |
|---|---|---|---|
| | (at the | 4° C. for | 25° C. for |
| Samples | beginning) | 11 weeks | 11 weeks |
| TPC (GAE mg/mL) | 7.637 ± 0.223 | 7.423 ± 0.173 | 7.411 ± 0.078 |

### Example 5

In this example, the effects of substituting water with other fruit juice in the mixture system of aronia juice and gum arabic were investigated and described.

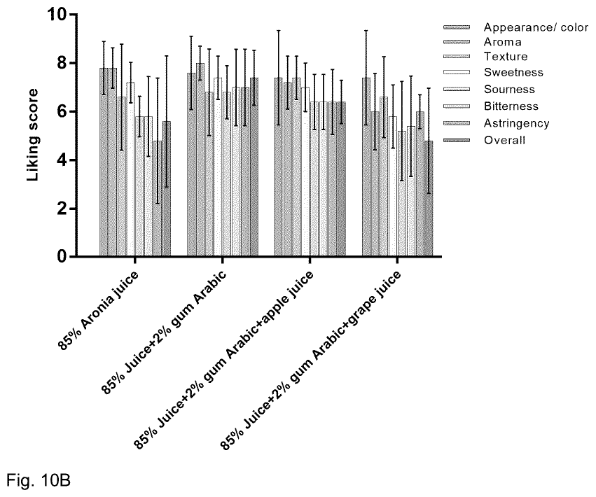

FIG. 10 indicated that aronia juice with 2.3% gum arabic (after transformation recipe) mixture system by supplementing the rest part with water was the most effective in attenuating the astringency intensity (FIG. 10(A)) and in improving the palatability (FIG. 10(B)) of aronia juice than supplementing with apple and grape juices, suggesting water is better than apple and grape juices in the mixture system of aronia juice with gum arabic.

### Example 6

In this example, the effect of gum arabic on the astringency intensity of aronia puree was described.

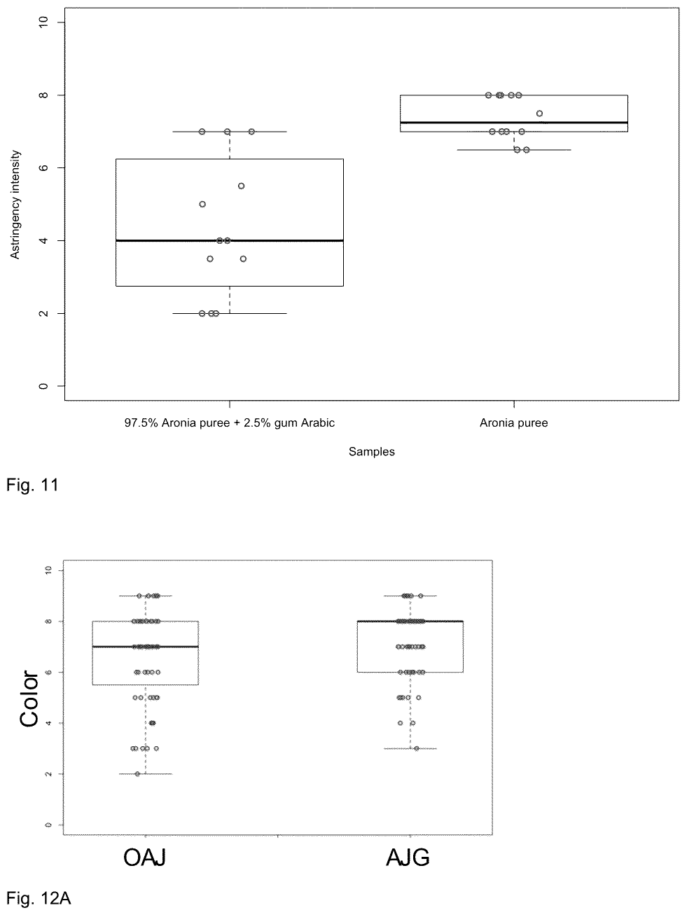

As shown in FIG. 11, introducing gum arabic into aronia puree system could also effectively attenuate the astringency intensity of aronia puree. Noteworthy, the pomace in aronia puree will bring a granular sensation in the oral cavity, which might be easy to be confounded with astringency sensation, as astringency was claimed as a tactile sensation by some people. Besides, when biting the puree, juice inside of the pomace will come out, which was not reacted with gum arabic, will probably trigger astringency sensation. Therefore, introducing gum arabic into aronia puree mainly restrained the polyphenol related astringency of free fluid juice in aronia puree.

### Example 7

In this example, aronia juice with 2% gum arabic was prepared in a pilot scale, and the effects of gum arabic on the organoleptic attributes of aronia juice were investigated and described.

According to Table 1, in order to use 2% gum arabic in aronia juice without adding water, aronia juice (2) and aronia juice (3) were blended to make a mixed aronia juice whose TPAC could meet the requirement. Here, 50% (w/w) of aronia juice (2) was mixed with 48% (w/w) aronia juice (3) and 2% (w/w) of gum arabic to make aronia juice with 2% gum. arabic (AJG). The same proportion (51:49, w/w) of aronia juice (2) and aronia juice (3) without gum arabic was mixed to make original aronia juice (OAJ). Both OAJ and AJG were treated with HPP at 600 MPa for 5 min. The ratio of gum arabic to TPAC in AJG was calculated as 25.35, which met the requirement that preferred at least 22.15-23.13.

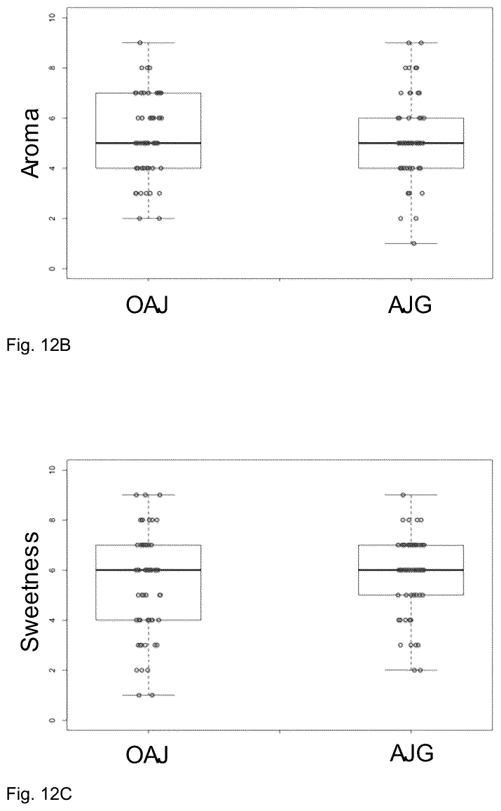

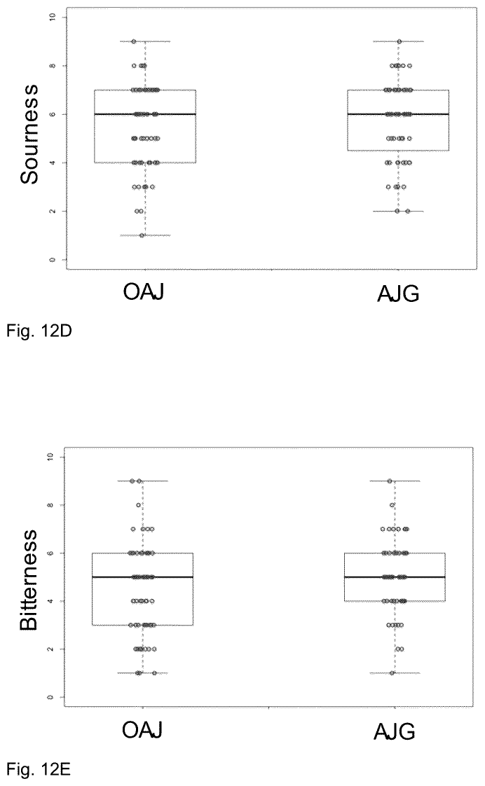

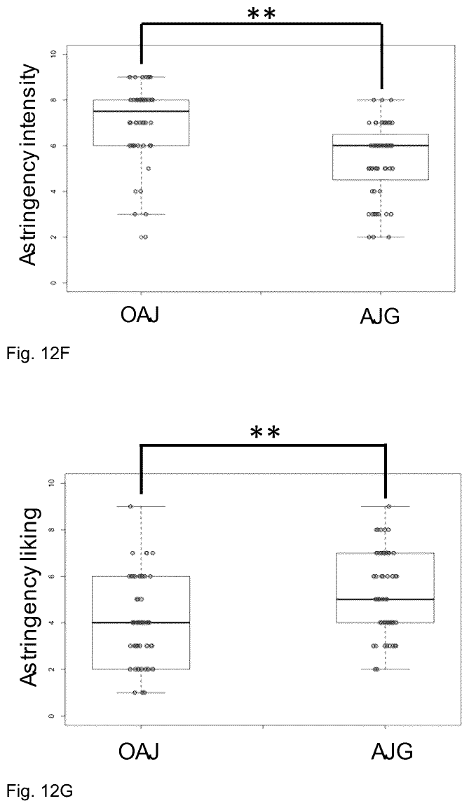

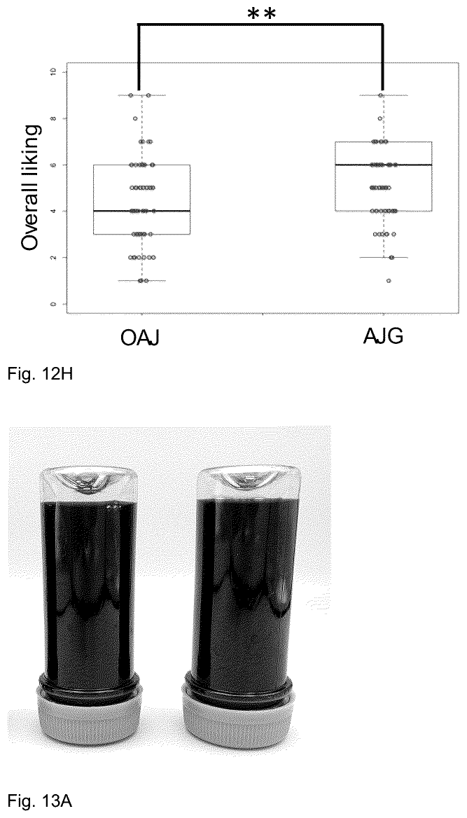

As displayed in FIG. 12, the addition of 2% gum arabic will not remarkably influence the hedonic towards color, aroma, sweetness, sourness, and bitterness of aronia juice, while the astringency intensity was significantly reduced from 7.12+1.57 (OAJ) to 5.33+1.57 (AJG), and the astringency liking (from 3.92+1.84 (OAJ) to 5.27+1.75 (AJG)) and overall liking (from 4.10+1.95 (OAJ) to 5.58+1.81 (AJG)) were both significantly improved. This indicated that the preferred ingredient and method developed in a lab scale were effective in a pilot scale.

### Example 8

In this example, the effects of gum arabic on the physicochemical properties of aronia juice were described.

As shown in Table 3 and Table 4, when comparing AJG (before HPP) to OAJ (before HPP), the addition of 2% gum arabic could increase the pH, total soluble solids content (° Brix), and ° Brix: acid ratio of aronia juice, but did not have significant effects on the titratable acidity and color.

TABLE 3
pH, total soluble solids content (°Brix), titratable acidity (%), and
°Brix: acid ratio of two types of aronia juice before and after high-
pressure processing (HPP) and being stored at 4° C. for 6 months (N = 3).

| | | | | |
|---|---|---|---|---|
| | | | Titratable | |
| Samples | pH | °Brix | acidity (%) | °Brix: acid |

Original aronia juice (OAJ)

| | | | | |
|---|---|---|---|---|
| OAJ (Before HPP) | 3.73 ± 0.02 | 17.28 ± 0.08 | 0.786 ± 0.003 | 21.991 ± 0.105 |
| OAJ (HPP, 0 M) | 3.70 ± 0.03 | 17.37 ± 0.08 | 0.777 ± 0.002 | 22.365 ± 0.143 |
| OAJ (HPP, 1 M) | 3.75 ± 0.02 | 17.11 ± 0.13 | 0.779 ± 0.003 | 21.969 ± 0.078 |
| OAJ (HPP, 2 M) | 3.71 ± 0.02 | 17.27 ± 0.03 | 0.778 ± 0.001 | 22.199 ± 0.045 |
| OAJ (HPP, 3 M) | 3.68 ± 0.03 | 17.12 ± 0.16 | 0.778 ± 0.005 | 21.995 ± 0.276 |
| OAJ (HPP, 4 M) | 3.74 ± 0.02 | 17.35 ± 0.05 | 0.780 ± 0.003 | 22.257 ± 0.064 |
| OAJ (HPP, 5 M) | 3.74 ± 0.01 | 17.05 ± 0.05 | 0.789 ± 0.004 | 21.601 ± 0.119 |
| OAJ (HPP, 6 M) | 3.79 ± 0.01 | 16.98 ± 0.03 | 0.776 ± 0.004 | 21.889 ± 0.104 |

Aronia juice with 2% gum arabic (AJG)

| | | | | |
|---|---|---|---|---|
| AJG (Before HPP) | 3.79 ± 0.01 | 19.00 ± 0.05 | 0.783 ± 0.002 | 24.255 ± 0.109 |
| AJG (HPP, 0 M) | 3.75 ± 0.02 | 19.02 ± 0.10 | 0.781 ± 0.003 | 24.335 ± 0.075 |
| AJG (HPP, 1 M) | 3.79 ± 0.01 | 19.00 ± 0.05 | 0.785 ± 0.002 | 24.202 ± 0.101 |
| AJG (HPP, 2 M) | 3.80 ± 0.01 | 18.95 ± 0.05 | 0.785 ± 0.002 | 24.132 ± 0.042 |
| AJG (HPP, 3 M) | 3.80 ± 0.03 | 18.97 ± 0.03 | 0.793 ± 0.002 | 23.925 ± 0.098 |
| AJG (HPP, 4 M) | 3.80 ± 0.01 | 19.10 ± 0.10 | 0.791 ± 0.001 | 24.132 ± 0.123 |
| AJG (HPP, 5 M) | 3.82 ± 0.03 | 19.02 ± 0.03 | 0.791 ± 0.003 | 24.053 ± 0.110 |
| AJG (HPP, 6 M) | 3.86 ± 0.02 | 18.88 ± 0.08 | 0.793 ± 0.000 | 23.820 ± 0.099 |

TABLE 4
Colorimetric analysis of two types of aronia juice
before and after high-pressure processing (HPP)
and being stored at 4° C. for 6 months (N = 3)

| | | | |
|---|---|---|---|
| Samples | L* | a* | b* |

Original aronia juice (OAJ)

| | | | |
|---|---|---|---|
| OAJ (Before HPP) | 35.39 ± 0.03 | 1.48 ± 0.06 | −0.55 ± 0.01 |
| OAJ (HPP, 0 M) | 35.31 ± 0.05 | 1.60 ± 0.07 | −0.54 ± 0.03 |
| OAJ (HPP, 1 M) | 35.36 ± 0.03 | 2.11 ± 0.11 | −0.46 ± 0.01 |
| OAJ (HPP, 2 M) | 35.39 ± 0.04 | 2.39 ± 0.10 | −0.39 ± 0.03 |
| OAJ (HPP, 3 M) | 35.38 ± 0.03 | 2.64 ± 0.04 | −0.35 ± 0.01 |
| OAJ (HPP, 4 M) | 35.44 ± 0.08 | 2.79 ± 0.22 | −0.32 ± 0.04 |
| OAJ (HPP, 5 M) | 35.53 ± 0.10 | 2.99 ± 0.04 | −0.28 ± 0.02 |
| OAJ (HPP, 6 M) | 35.43 ± 0.10 | 2.87 ± 0.01 | −0.32 ± 0.01 |

Aronia juice with 2% gum arabic (AJG)

| | | | |
|---|---|---|---|
| AJG (Before HPP) | 35.43 ± 0.07 | 1.42 ± 0.07 | −0.58 ± 0.02 |
| AJG (HPP, 0 M) | 35.32 ± 0.03 | 1.48 ± 0.04 | −0.55 ± 0.01 |
| AJG (HPP, 1 M) | 35.34 ± 0.02 | 1.39 ± 0.01 | −0.57 ± 0.02 |
| AJG (HPP, 2 M) | 35.35 ± 0.01 | 1.41 ± 0.03 | −0.54 ± 0.01 |
| AJG (HPP, 3 M) | 35.37 ± 0.01 | 1.48 ± 0.07 | −0.53 ± 0.03 |
| AJG (HPP, 4 M) | 35.36 ± 0.02 | 1.50 ± 0.05 | −0.49 ± 0.01 |
| AJG (HPP, 5 M) | 35.40 ± 0.05 | 1.61 ± 0.01 | −0.51 ± 0.01 |
| AJG (HPP, 6 M) | 35.34 ± 0.02 | 1.54 ± 0.05 | −0.48 ± 0.02 |

L*: lightness. 0 = black, 100 = white,
a*: redness. (−a*) greenness, (±a*) redness,
b*: blueness. (−b*) blueness to (±b*) yellowness.

### Example 9

In this example, the microbial analyses for original aronia juice and aronia juice with 2% gum arabic during shelf-life storage for 6 months were described.

As shown in Table 5, after HPP treatment, the APC, yeast, and mold of both OAJ and AJG were decreased to <1 log CFU/mL. During the storage at 4° C. for 6 months, the APC, yeast, and mold were always kept at below 1 log CFU/mL, which indicated the safety of both products.

TABLE 5
Total aerobic plate counts (APC), yeast, and mold in two types
of aronia juice before and after high-pressure processing
(HPP) and being stored at 4° C. for 6 months (N = 3).

| | | | |
|---|---|---|---|
| | APC | Yeast | Mold |
| Samples | (log CFU/mL) | (log CFU/mL) | (log CFU/mL) |

Original aronia juice (OAJ)

| | | | |
|---|---|---|---|
| OAJ (Before HIPP) | 4.660 ± 0.198 | 4.772 ± 0.209 | <1 |
| OAJ (HPP, 0 M) | <1 | <1 | <1 |
| OAJ (HPP, 1 M) | <1 | <1 | <1 |
| OAJ (HPP, 2 M) | <1 | <1 | <1 |
| OAJ (HPP, 3 M) | <1 | <1 | <1 |
| OAJ (HPP, 4 M) | <1 | <1 | <1 |
| OAJ (HPP, 5 M) | <1 | <1 | <1 |
| OAJ (HPP, 6 M) | <1 | <1 | <1 |

Aronia juice with 2% gum arabic (AJG)

| | | | |
|---|---|---|---|
| AJG (Before HPP) | 4.373 ± 0.127 | 4.482 ± 0.092 | <1 |
| AJG (HPP, 0 M) | <1 | <1 | <1 |
| AJG (HPP, 1 M) | <1 | <1 | <1 |
| AJG (HPP, 2 M) | <1 | <1 | <1 |
| AJG (HPP, 3 M) | <1 | <1 | <1 |
| AJG (HPP, 4 M) | <1 | <1 | <1 |
| AJG (HPP, 5 M) | <1 | <1 | <1 |
| AJG (HPP, 6 M) | <1 | <1 | <1 |

### Example 10

In this example, the stability of original aronia juice and aronia juice with 2% gum arabic during shelf-life storage for 6 months were described.

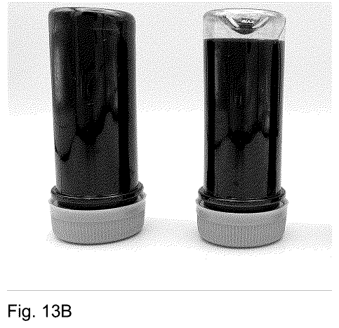

FIG. 13 showed the addition of gum arabic could obviously prevent the formation of precipitate and flocculation and enhance the physical stability of aronia juice during storage.

During the 6 months storage, for both OAJ and AJG, the pH value and titratable acidity showed an increasing trend, while the ° Brix and ° Brix: acid ratio showed a decreasing trend (Table 3). The color of OAJ was significantly changed to be redder and yellower with time going by, while the addition of 2% gum arabic could effectively prevent the color change of OAJ (Table 4). After being stored at 4° C.for 6 months, the total phenolic content (TPC) and total anthocyanin content (TAC) of OAJ were decreased by 12.92% and 41.10%, respectively, while the TPAC was increased by 1.10%. As for the AJG, the TPC, TPAC, and TAC were decreased by 7.78%, 6.03%, and 32.33%, respectively (Table 6), indicating the addition of gum arabic could effectively prevent the anthocyanins from degradation or being polymerized to form proanthocyanidins.

TABLE 6
Phenolic profile of two types of aronia juice before
and after high-pressure processing (HPP) and being
stored at 4° C. for 6 months (N = 3).

| | | | |
|---|---|---|---|
| | TPC (mg | TPAC (mg | TAC (mg |
| Samples | GAE/mL) | CE/mL) | Cy3GlcE/mL) |

Original aronia juice (OAJ)

| | | | |
|---|---|---|---|
| OAJ (Before HPP) | 4.762 ± 0.274 | 0.519 ± 0.058 | 1.206 ± 0.108 |
| OAJ (HPP, 0 M) | 5.006 ± 0.208 | 0.635 ± 0.030 | 1.185 ± 0.071 |
| OAJ (HPP, 1 M) | 4.814 ± 0.211 | 0.622 ± 0.043 | 1.025 ± 0.072 |
| OAJ (HPP, 2 M) | 4.733 ± 0.351 | 0.620 ± 0.017 | 0.938 ± 0.044 |
| OAJ (HPP, 3 M) | 4.455 ± 0.223 | 0.614 ± 0.080 | 0.858 ± 0.038 |
| OAJ (HPP, 4 M) | 4.511 ± 0.285 | 0.558 ± 0.040 | 0.810 ± 0.050 |
| OAJ (HPP, 5 M) | 4.684 ± 0.234 | 0.599 ± 0.051 | 0.778 ± 0.059 |
| OAJ (HPP, 6 M) | 4.359 ± 0.164 | 0.642 ± 0.039 | 0.698 ± 0.024 |
| Change (%, 6 M | −12.92% | +1.10% | −41.10% |
| to 0 M) | | | |

Aronia juice with 2% gum arabic (AJG)

| | | | |
|---|---|---|---|
| AJG (Before HPP) | 5.676 ± 0.290 | 0.415 ± 0.053 | 1.089 ± 0.060 |
| AJG (HPP, 0 M) | 5.846 ± 0.196 | 0.448 ± 0.021 | 1.101 ± 0.075 |
| AJG (HPP, 1 M) | 5.584 ± 0.212 | 0.438 ± 0.036 | 1.048 ± 0.096 |
| AJG (HPP, 2 M) | 5.724 ± 0.202 | 0.398 ± 0.040 | 0.942 ± 0.043 |
| AJG (HPP, 3 M) | 5.484 ± 0.252 | 0.406 ± 0.023 | 0.924 ± 0.020 |
| AJG (HPP, 4 M) | 5.399 ± 0.122 | 0.356 ± 0.039 | 0.873 ± 0.036 |
| AJG (HPP, 5 M) | 5.528 ± 0.068 | 0.440 ± 0.017 | 0.825 ± 0.037 |
| AJG (HPP, 6 M) | 5.391 ± 0.265 | 0.421 ± 0.016 | 0.745 ± 0.023 |
| Change (%, 6 M | −7.78% | −6.03% | −32.33% |
| to 0 M) | | | |

### Objectives

1. To provide suitable ingredients and additive amount for mitigating the astringency intensity and increasing the palatability of aronia berry; (ingredients)
2. To provide suitable processing procedures for introducing the ingredients into aronia berry compositional system; (methods)
3. To improve the physical stability and protect/maintain the phenolic compounds of aronia berry juice products by introducing suitable ingredients and providing proper processing procedures. (effects)
4. To apply the preferred ingredient and processing method to prepare modified aronia juice in a pilot scale, and the changes in sensory properties were evaluated by a large size of sensory panel.
5. To determine the changes in physicochemical properties of aronia juice after introducing gum arabic.
6. To compare the stabilities of original aronia juice (OAJ) and aronia juice with 2% gum arabic (AJG) during the storage at 4° C.for 6 months.

### Materials and Methods

### 1.1. Materials

Aronia berries (A. melanocarpa ‘Viking’) were harvested from two farms located in NE. Pectin from Profood Products Outlet was bought on eBay. κ-carrageenan (from Modernist Pantry), xanthan gum (from Anthony's), sodium carboxymethyl cellulose (CMC-Na, from Eisen-Golden Laboratories), and gum arabic from Pure Supplements, were bought on Amazon.

Six concentrations of polysaccharides including pectin, κ-carrageenan, xanthan gum, CMC-Na, gum arabic (0.1, 0.5, 1, 1.5, 2, 2.5, 3%, w/w) were selected to be introduced into aronia juice by mixing vigorously using vortex. The total weight of mixture is set as 10 g, and the reaction was triggered in a 15 mL centrifuge tube. After being centrifuged, all tubes were inverted and compared to the tube containing original aronia juice (pictures were taken to display the physical status/appearance). Less precipitate the reaction forms, more suitable the ingredient is for aronia juice.

Concentrations of gum arabic including 1, 1.2, 1.4, 1.6, 1.8, 2.0, 2.1, 2.2, 2.3, 2.4, 2.5, 2.6, 2.7, 2.8, 2.9, 3.0% (w/w) were selected to be introduced into aronia juice in the same way mentioned in the section 1.2. At each concentration point, physical status/appearance (reflected by pictures), turbidity, total phenolic content (TPC, by Folin-Ciocalteu method), and total anthocyanins content (TAC, by pH differential method) were tested and compared.

Four well-trained panelists were invited to participate in the sensory panel to further determine the minimal concentration of gum arabic (2-2.6%) that should be added to effectively mitigate the astringency and increase the palatability of aronia juice. According to the FDA's policy, 2% of gum arabic is the maximum concentration allowed to be used in beverage bases. Therefore, we transformed the gum arabic concentration to 2% in each system and fixed the ratio of gum arabic to aronia juice, the insufficient part was supplemented by water. The transformation recipe was shown in Table 7.

TABLE 7
Transformation recipe for turning
gum arabic (GA) concentration to 2%

| GA | | |
|---|---|---|
| concen- | | |
| tration (%) | Before transformation | After transformation |
| 2% | 2% GA + 98% Juice | 2% GA + 98% Juice |
| 2.1% | 2.1% GA + 97.9% juice | 2% GA + 4.76% water + |
| | | 93.24% juice |
| 2.2% | 2.2% GA + 97.8% juice | 2% GA + 9.1% water + 88.9% |
| | | juice |
| 2.3% | 2.3% GA + 97.7% juice | 2% GA + 13% water + 85% juice |
| 2.4% | 2.4% GA + 97.6% juice | 2% GA + 16.7% water + 81.3% |
| | | juice |
| 2.5% | 2.5% GA + 97.5% juice | 2% GA + 20% water + 78% juice |
| 2.6% | 2.6% GA + 97.4% juice | 2% GA + 23.08% water + |
| | | 74.92% juice |

The supernatant of samples (aronia juice and the mixtures of aronia juice with different concentrations of gum arabic) after centrifugation were stored in the fridge for a week (7 days). The physical status/appearance was recorded before and after storage. The appearances were compared for getting more narrow and suitable range of gum arabic concentrations. Sensory panel was performed again based on the suitable concentrations of gum arabic obtained in this storage stability section to validate the effects on the astringency intensity and palatability of aronia juice.

The introducing procedures of gum arabic into aronia juice system played an important role in avoiding flocculation when mixing. Adding gum arabic to the aronia juice with very fast and vigorous mixing has been proved to be successful in preparing modified aronia juice. In this section, we firstly compared “adding gum arabic to aronia juice without very fast and vigorously mixing” with “adding aronia juice slowly to gum arabic with stirring”. Secondly, we compared “dissolving gum arabic in the water first and then adding aronia juice” with “adding aronia juice slowly to gum arabic with stirring and then adding water”.

The storage stability of aronia juice with and without suitable concentration of gum arabic after being pasteurized was investigated at different temperatures (4° C. and 25° C.) when being stored for 1 and 11 weeks. The physical status/appearance and TPC of each sample were compared.

To investigate the effects of blending other fruit juice on the improvement of astringency and palatability of aronia juice, we compared “85% aronia juice, 13% water with 2% gum arabic” with “85% aronia juice with 15% water”, “85% aronia juice, 13% apple juice with 2% gum arabic”, and “85% aronia juice, 13% grape juice with 2% gum arabic”.

The astringency intensity of “97.5% aronia berry puree with 2.5% gum arabic (w/w)” was compared with “100% aronia berry puree”.

OAJ and AJG were prepared in a pilot scale based on the preferred method developed in the lab scale. All samples were treated with HPP at 600 MPa for 5 min, and stored at 4° C.for 6 months. During the storage, physicochemical properties including physical status/appearance, pH, total soluble solids content (° Brix), titratable acidity (%), ° Brix: acid ratio, and color were monitored. TPC, TPAC, and TAC were determined. Total aerobic plate counts (APC), yeast, and mold in samples were tested.

### Discussion

This disclosure determined that:
1) Gum arabic is preferred ingredient to be introduced into aronia juice system for mitigating the astringency intensity and increasing the palatability.
2) The preferred added amount of gum arabic is based on the ratio of gum arabic to total proanthocyanidin content (TPAC) (w/w) in aronia juice. The preferred ratio is at least 22.15-23.13, i.e., if the TPAC in aronia juice was determined as 1 g CE by DMAC method, at least 22.15-23.13 g of gum arabic should be used. Of course, higher amounts can also be used. For example, 23.2, 23.3, 23.4, 23.5, 23.6, 23.7. 23.8, 23.9. 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.5, 27, 27.5, 28, 28.5, 29, 29.5, 30, and the like.
3) The preferred processing methods include “adding gum arabic to the aronia juice with very fast and vigorous mixing”, or “adding aronia juice slowly and gradually to the gum arabic with continuous stirring”, or “dissolving gum arabic in the water first and then adding aronia juice gradually with stirring”.
4) During storage, gum arabic could effectively enhance the physical stability and protect/maintain the phenolic compounds from degradation or flocculation in aronia juice.
5) Substituting water with apple juice or grape juice in the mixture system of aronia juice with gum arabic could not improve the effects on the astringency intensity and palatability.
6) Introducing gum arabic into aronia puree effectively restrained the polyphenol related astringency of free fluid juice in aronia puree.
7) In a pilot scale, addition of gum arabic was validated to effectively reduce the astringency intensity and improve overall liking of aronia juice.
8) The addition of 2% gum arabic could increase the pH, total soluble solids content (° Brix), and ° Brix: acid ratio of aronia juice, but did not have significant effects on the titratable acidity and color.
9) During the 6 months shelf-life storage, HPP-treated aronia juice with gum arabic was safe in terms of the total aerobic counts (APC), yeast, and mold (<1 log CFU/mL).
10) During storage, the addition of gum arabic could effectively enhance the physical stability and prevent the aronia juice from changing the color to be redder and yellower with time going by.
11) The addition of gum arabic could effectively prevent the anthocyanins from degradation or being polymerized to form proanthocyanidins.

What is claimed is:

1. A composition comprising aronia berry juice and gum arabic.

2. The composition of claim 1 wherein the aronia berry juice is present in the composition in an amount of greater than 10% w/w.

3. The composition of claim 2, wherein the aronia berry juice comprises at least 75% w/w of the composition.

4. The composition of claim 1, wherein the composition comprises up to 4% w/w gum arabic.

5. The composition of claim 1, wherein the ratio of gum arabic to total proanthocyanidins content (TPAC) (w/w) in said aronia berry juice is at least 22.15.

6. A method of reducing the astringency of an aronia berry composition comprising the step of combining the composition with an amount of gum arabic.

7. The method of claim 6, wherein the aronia berry composition and the gum arabic are combined using a method selected from the group consisting of adding the aronia berry composition slowly to gum arabic by stirring, adding gum arabic to the aronia berry composition with very fast and vigorous mixing, adding the aronia berry composition slowly and gradually to the gum arabic with continuous stirring, or dissolving gum arabic in the water first and then adding aronia berry composition gradually with stirring.

8. The method of claim 6, wherein the gum arabic is in a form selected from the group consisting of powder or aqueous liquid.

9. The method of claim 6, wherein the aronia berry composition is aronia berry juice.

10. The method of claim 9 wherein the aronia berry juice is present in the composition in an amount of greater than 75% w/w.

11. The method of claim 9, wherein the aronia berry juice comprises 84-86% w/w of the composition.

12. The method of claim 6, wherein the aronia berry composition comprises up to 4% w/w gum arabic.

13. The method of claim 6, wherein the ratio of gum arabic to total proanthocyanidins content (TPAC) (w/w) in said aronia berry juice is at least 22.15.

14. A method of increasing the stability of an aronia berry composition comprising the step of combining the composition with an amount of gum arabic.

15. The method of claim 14, wherein the aronia berry composition is added to gum arabic using a method selected from the group consisting of adding the aronia berry composition slowly to gum arabic by stirring, adding gum arabic to the aronia berry composition with very fast and vigorous mixing, adding the aronia berry composition slowly and gradually to the gum arabic with continuous stirring, or dissolving gum arabic in the water first and then adding aronia berry composition gradually with stirring

16. The method of claim 14, wherein the gum arabic is in a form selected from the group consisting of powder and aqueous liquid.

17. The method of claim 14, wherein the aronia berry composition is aronia berry juice.

18. The method of claim 17, wherein the aronia berry juice is present in the composition in an amount of greater than 75% w/w.

19. The method of claim 14, wherein the aronia berry juice comprises 84-86% w/w of the composition.

20. The method of claim 14, wherein the composition comprises up to 4% w/w gum arabic.

21. The method of claim 14, wherein the ratio of gum arabic to total proanthocyanidins content (TPAC) (w/w) in said aronia berry juice is at least 22.15.

22. The method of claim 14, wherein the increase in stability is determined by a comparison of the TPAC between an aronia berry composition containing gum arabic and an aronia berry composition that does not contain gum arabic.

23. The method of claim 14, wherein the increase in stability is determined by a comparison of the amount of aggregation or flocculation between an aronia berry composition that contains gum arabic and an aronia berry composition that does not contain gum arabic.

24. The method of claim 14, wherein the increase in stability is determined by a comparison of the content of at least one nutrient between an aronia berry composition that contains gum arabic and an aronia berry composition that does not contain gum arabic.
